(19) United States
(12) Patent Application Publication
(10) Pub. No.: US 2005/0014959 A1
(43) Pub. Date: Jan. 20, 2005

(54) Method for the production of dihydroxycarboxylate esters

(76) Inventors: Martin Jochen Klatt, Bad Durkheim (DE); Markus Niebel, Mannheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/495,427

(22) PCT Filed: Dec. 5, 2002
(86) PCT No.: PCT/EP02/13774

(30) Foreign Application Priority Data
Dec. 7, 2001 (DE) 101 60 148.4

(51) Int. Cl. C07C 69/66; C07D 307/02

(52) U.S. Cl. 549/475; 560/186

(57) ABSTRACT

A description is given of a process for preparing dihydroxycarboxylic esters and of an overall process for preparing R-(+)-α-lipoic acid.

The present invention relates to a process for preparing dihydroxycarboxylic esters and an overall process for preparing R-(+)-α-lipoic acid.

Dihydroxycarboxylic esters are valuable intermediates and synthesis building blocks in organic chemistry. In particular, (6S)-6,8-dihydroxyoctanoic esters serve as intermediates for the synthesis of enantiomerically pure R-(+)-α-lipoic acid.

EP 487 986 discloses preparing (6S)-6,8-dihydroxyoctanoic esters by reducing the corresponding (3S)-3-hydroxyoctanedioic diesters with complex hydrides in the presence of aprotic solvents.

Using this process, yields which are good but are still in need of improvement are achieved. In addition, the process has the disadvantages that relatively large amounts of complex hydrides must be used.

It is an object of the present invention, therefore, to provide a process for preparing dihydroxycarboxylic esters which does not have the disadvantages of the prior art and provides the dihydroxycarboxylic esters in improved yields.

We have found that this object is achieved by a process for preparing dihydroxycarboxylic esters of the formula I, 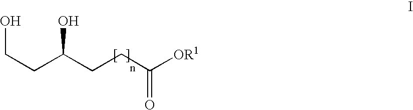
where
n is 1, 2, 3, 4, 5, 6 or 7 and
R¹ is an unsubstituted or substituted C₁-C₂₀-alkyl, C₂-C₂₀-alkenyl, C₂-C₂₀-alkynyl, C₃-C₈-cycloalkyl, aralkyl, aryl, hetarylalkyl or hetaryl radical,
   which comprises reacting hydroxycarboxylic diesters of the formula II, 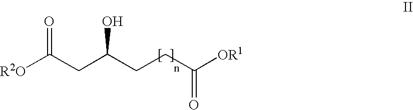
   where
R² is a radical R¹ independent of R¹,
   with complex hydrides in the presence of a solvent and in the presence of phase transfer catalysts.

The index n which is the number of —CH₂— radicals is 1, 2, 3, 4, 5, 6 or 7, preferably 3. In a preferred embodiment of the process, therefore dihydroxyoctanoic esters are prepared according to the invention.

The radicals R¹ and R² can be different or identical. The radicals R¹ and R² are therefore independently of one another an unsubstituted or substituted C₁-C₂₀-alkyl, preferably C₁-C₁₂-alkyl, particularly preferably C₁-C₄-alkyl, an unsubstituted or substituted C₂-C₂₀-alkenyl, preferably C₂-C₁₂-alkenyl, particularly preferably C₁-C₄-alkenyl, an unsubstituted or substituted C₂-C₂₀-alkynyl, preferably C₂-C₁₂-alkynyl, particularly preferably C₁-C₄-alkynyl, an unsubstituted or substituted C₃-C₈-cycloalkyl, an unsubstituted or substituted aralkyl, an unsubstituted or substituted aryl, an unsubstituted or substituted hydroxyalkyl or an unsubstituted or substituted hetaryl.

For all substituted radicals of the present invention, if the substituents are not specified in more detail, independently of one another there may be up to five substituents, for example selected from the following group:
halogen, in particular F or Cl, unsubstituted or substituted C₁-C₁₂-alkyl, in particular C₁-C₄-alkyl, for example methyl, CF₃, C₂F₅ or CH₂F or C₁-C₁₂-alkoxy, in particular C₁-C₄-alkoxy.
C₁-C₁₂-Alkyl radicals for R¹ and R² are independently of one another, for example, methyl, ethyl, propyl, 1-methylethyl, butyl, 1-methylpropyl, 2-methylpropyl, 1,1-dimethylethyl, pentyl, 1-methylbutyl, 2-methylbutyl, 1,2-dimethylpropyl, 1,1-dimethylpropyl, 2,2-dimethylpropyl, 1-ethylpropyl, hexyl, 1-methylpentyl, 1,2-dimethylbutyl, 1,3-dimethylbutyl, 2,3-dimethylbutyl, 1,1-dimethylbutyl, 2,2-dimethylbutyl, 3,3-dimethylbutyl, 1,1,2-trimethylpropyl, 1,2,2-trimethylpropyl, 1-ethylbutyl, 2-ethylbutyl, 1-ethyl-2-methylpropyl, heptyl, octyl, nonyl, decyl, undecyl or dodecyl, preferably branched or unbranched C₁-C₄-alkyl radicals, for example methyl, ethyl, propyl, 1-methylethyl, butyl, 1-methylpropyl, 2-methylpropyl or 1,1-dimethylethyl, particularly preferably methyl.

A C₂-C₁₂-alkenyl radical for R¹ and R² is, independently of one another, for example, vinyl, 2-propenyl, 2-butenyl, 3-butenyl, 1-methyl-2-propenyl, 2-methyl-2-propenyl, 2-pentenyl, 3-pentenyl, 4-pentenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-2-propenyl, 1-ethyl-2-propenyl, 2-hexenyl, 3-hexenyl, 4-hexenyl, 5-hexenyl, 1-methyl-2-pentenyl, 2-methyl-2-pentenyl, 3-methyl-2-pentenyl, 4-methyl-2-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-2-butenyl, 1,2-dimethyl-3-butenyl, 1,3-dimethyl-2-butenyl, 1,3-dimethyl-3-butenyl, 2,2-dimethyl-3-butenyl, 2,3-dimethyl-2-butenyl, 2,3-dimethyl-3-butenyl, 1-ethyl-2-butenyl, 1-ethyl-3-butenyl, 2-ethyl-2-butenyl, 2-ethyl-3-butenyl, 1,1,2-trimethyl-2-propenyl, 1-ethyl-l-methyl-2-propenyl, 1-ethyl-2-methyl-2-propenyl and the corresponding heptenyls, octenyls, nonenyls, decenyls, undecenyls and dodecenyls.

A C₂-C₁₂-alkynyl radical for R¹ and R² is, independently of one another, for example, ethynyl, 2-propynyl, 2-butynyl, 3-butynyl, 1-methyl-2-propynyl, 2-pentynyl, 3-pentynyl, 4-pentynyl, 1-methyl-3-butynyl, 2-methyl-3-butynyl, 1-methyl-2-butynyl, 1,1-dimethyl-2-propynyl, 1-ethyl-2-propynyl, 2-hexynyl, 3-hexynyl, 4-hexynyl, 5-hexynyl, 1-methyl-2-pentynyl, 1-methyl-2-pentynyl, 1-methyl-3-pentynyl, 1-methyl-4-pentynyl, 2-methyl-3-pentynyl, 2-methyl-4-pentynyl, 3-methyl-4-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 1-ethyl-2-butynyl, 1-ethyl-3-butynyl, 2-ethyl-3-butynyl and 1-ethyl-1-methyl-2-propynyl, preferably ethynyl, 2-propynyl, 2-butynyl, 1-methyl-2-propynyl or 1-methyl-2-butynyl, and the corresponding heptynyls, octynyls, nonynyls, decynyls, undecynyls and dodecynyls.

A C₃-C₈-cycloalkyl radical for R¹ and R² is, independently of one another, for example, cyclopropyl, cyclobutyl, cyclopentyl, cyclohexyl, cycloheptyl or cyclooctyl.

Preferred unsubstituted or substituted aryl radicals for R₁ and R₂ are, independently of one another, unsubstituted or substituted phenyl, 1-naphthyl or 2-naphthyl.

Preferred unsubstituted or substituted arylalkyl radicals for R₁ and R₂ are, independently of one another, unsubstituted or substituted benzyl or ethylenephenyl (homobenzyl).

Hetaryl radicals for R¹ and R² are, independently of one another, for example radicals such as 2-pyridyl, 3-pyridyl, 4-pyridyl, 2-furyl, 3-furyl, 2-pyrrolyl, 3-pyrrolyl, 2-thienyl, 3-thienyl, 2-thiazolyl, 4-thiazolyl, 5-thiazolyl, 2-oxazolyl, 4-oxazolyl, 5-oxazolyl, 2-pyrimidyl, 4-pyrimidyl, 5-pyrimidyl, 6-pyrimidyl, 3-pyrazolyl, 4-pyrazolyl, 5-pyrazolyl, 3-isothiazolyl, 4-isothiazolyl, 5-isothiazolyl, 2-imidazolyl, 4-imidazolyl, 5-imidazolyl, 3-pyridazinyl, 4-pyridazinyl, 5-pyridazinyl, 6-pyridazinyl, 3-isoxazolyl, 4-isoxazolyl, 5-isoxazolyl, thiadiazolyl, oxadiazolyl or triazinyl.

Substituted hetaryl radicals R¹ and R² are, independently of one another, also anellated derivatives of the abovementioned hetaryl radicals, for example indazole, indole, benzothiophene, benzofuran, indoline, benzimidazole, benzthiazole, benzoxazole, quinoline, 2,3-dihydro-1-benzofuran, furo[2,3]pyridine, furo[3,2]pyridine or isoquinoline.

Hetarylalkyl radicals for R¹ and R² are, independently of one another, radicals which are composed, for example, of C₁-C₆-alkylene radicals and of the above-described hetaryl radicals, for example the radicals —CH₂-2-pyridyl, —CH₂-3-pyridyl, —CH₂-4-pyridyl, —CH₂-2-thienyl, —CH₂-3-thienyl, —CH₂-2-thiazolyl, —CH₂-4-thiazolyl, CH₂-5-thiazolyl, —CH₂-CH₂-2-pyridyl, —CH₂-CH₂-3-pyridyl, —CH₂-CH₂-4-pyridyl, —CH₂-CH₂-2-thienyl, —CH₂-CH₂-3-thienyl, —CH₂-CH₂-2-thiazolyl, —CH₂-CH₂-4-thiazolyl, or —CH₂-CH₂-5-thiazolyl.

Preferred radicals for R¹ and R² are, independently of one another, unsubstituted radicals. Particularly preferred radicals for R¹ and R² are, independently of one another, the above-described C₁-C₁₂-alkyl radicals, in particular C₁-C₄-alkyl, in particular methyl.

In a particularly preferred embodiment, the radicals R¹ and R² are identical.

The starting compounds of the inventive process are the hydroxycarboxylic diesters of the formula II. Preparation of these starting compounds is known per se and is described, for example, in EP 487 986 and in the references cited therein. Preferred hydroxycarboxylic diesters of the formula II as starting compounds are composed of the above-described preferred radicals R¹ and R² and the preferred index n.

Particularly preferred hydroxycarboxylic diesters of the formula II as starting compound are:
dimethyl (3S)-3-hydroxyoctanedioate,
1-ethyl 8-methyl (3S)-3-hydroxyoctanedioate,
8-methyl 1-propyl (3S)-3-hydroxyoctanedioate,
8-methyl 1-isopropyl (3S)-3-hydroxyoctanedioate,
1-butyl 8-methyl (3S)-3-hydroxyoctanedioate,
1-sec-butyl 8-methyl (3S)-3-hydroxyoctanedioate,
8-methyl 1-tert-butyl (3S)-3-hydroxyoctanedioate,
8-methyl 1-octyl (3S)-3-hydroxyoctanedioate,
8-methyl 1-phenyl (3S)-3-hydroxyoctanedioate and
1-(2-ethylhexyl) 8-methyl (3S)-3-hydroxyoctanedioate.

A particularly preferred starting compound is dimethyl (3S)-3-hydroxyoctanedioate.

Dihydroxycarboxylic esters of the formula I are prepared inventively as product compounds by reacting hydroxycarboxylic diesters of the formula II as starting compounds with complex hydrides and thus by reducing the hydroxycarboxylic diesters of the formula II as starting compounds in the presence of a solvent and in the presence of phase transfer catalysts.

Preferred complex hydrides are borohydrides, in particular ammonium borohydride, lithium borohydride, potassium borohydride and sodium borohydride, and also alkyl- and alkoxy-substituted borohydrides, for example lithium triethylborohydride and sodium trimethoxyborohydride. A particularly preferred complex hydride in the inventive process is sodium borohydride.

The molar ratio of complex hydrides to the hydroxycarboxylic diester of the formula II is not critical and is typically from 0.5:1 to 3:1, preferably from 0.5:1 to 1.5:1.

Preferred aprotic solvents are aliphatic and aromatic hydrocarbons, for example hexane, cyclohexane, toluene, benzene and xylene, and also ethers, for example dioxane, diethyl ether and tetrahydrofuran.

Particularly preferred aprotic solvents are aliphatic and aromatic hydrocarbons, for example hexane, cyclohexane, toluene, benzene and xylene, and very particularly preferred toluene.

Phase transfer catalysts are compounds which in a manner known per se are able to increase the exchange of compounds between at least two phases of the phase boundary, also termed the interphase. This can be a liquid/liquid or a liquid/solid phase boundary.

For the inventive process, in principle, all phase transfer catalysts are suitable.

Preference is given to phase transfer catalysts which are able to increase the exchange of compounds between an organic phase and an aqueous phase at the phase boundary. These phase transfer catalysts are described, for example, in E. V. Dehmlov, S. S. Dehmlov, Phase Transfer Catalysis, 3rd Edition, V C H Weinheim 1993, pages 65 to 71, in particular page 65.

Particularly preferred phase transfer catalysts are ammonium salts of the formula III
R³R⁴R⁵R⁶N⁺X⁻ III
where
R³, R⁴, R⁵ and R⁶ independently of one another are an unsubstituted or substituted aliphatic C₁-C₃₀ radical or an unsubstituted or substituted aralkyl or aryl radical and
X⁻ is a counterion,
and also the analogous phosphonium salts, for example tributylhexadecylphosphonium bromide, ethyltriphenylphosphonium bromide, tetraphenylphosphonium chloride, benzyltriphenylhosphonium iodide and tetrabutylphosphonium chloride.

In a preferred embodiment, the phase transfer catalysts used are ammonium salts of the formula III.

The radicals R³, R⁴, R⁵ and R⁶ can be identical or different.

An aliphatic C₁-C₃₀-radical is preferably a C₁-C₃₀-alkyl, C₂-C₃₀-alkenyl, C₂-C₃₀-alkynyl or C₃-C₈-cycloalkyl radical.

Aralkyl or aryl radicals for R³, R⁴, R⁵ and R⁶ are preferably the corresponding radicals described above for R¹ and R².

The type of counterion X⁻ is not critical; preferred counterions X⁻ are halide, hydrogensulfate or hydroxide.

Particularly preferred phase transfer catalysts are tricaprylmethylammonium chloride which is commercially available as Aliquat 336^{(R)}, for example from Fluka or Adogen 464^{(R)}, for example from Aldrich, benzyltriethylammonium chloride or benzyltriethylammonium bromide, tetrabutylammonium chloride or tetrabutylammonium bromide or cetyltrimethylarmonium chloride or cetyltrimethylammonium bromide.

The amount of phase transfer catalyst is not critical and is typically from 0.1 to 20 mol %, based on the hydroxycarboxylic diester of the formula II.

The temperature at which the inventive process is carried out is not critical and is typically from 0 to 150° C., preferably from 20 to 110° C. The inventive process is typically carried out at atmospheric pressure, but can also be carried out at reduced or slightly elevated pressure, preferably at from 0.1 to 10 bar. The reaction times are not critical and are typically from 0.5 to 5 hours, in particular from 1 to 3 hours.

In order to speed up the process of the invention, it is advantageous to add catalytic amounts of methanol to the reaction mixture.

The dihydroxycarboxylic esters of the formula I are isolated in a manner known per se, for example, by working up the reaction mixture by hydrolysis, extraction and drying.

The inventive process has the advantage that the dihydroxycarboxylic esters of the formula I can be prepared in high yields and using relatively small amounts of complex hydrides.

The invention further relates to an overall process for preparing R-(+)-α-lipoic acid using the inventive process as an intermediate step.

The invention therefore relates to a process for preparing R-(+)-α-lipoic acid of the formula IV 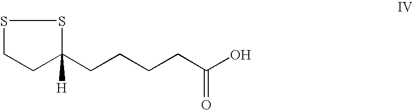
which comprises preparing dihydroxycarboxylic esters of the formula I, where n is 3, by reacting the corresponding hydroxycarboxylic diesters of the formula II with complex hydrides in the presence of a solvent and in the presence of phase transfer catalysts and, in a manner known per se,
a) converting these dihydroxycarboxylic esters of the formula I in organic solution using a sulfonyl chloride and a tertiary nitrogen base into the bissulfonic esters of I,
b) reacting these bissulfonic esters with sulfur and an alkali metal disulfide in a polar solvent to give the R-a-lipoic ester and
c) converting this ester into the R-(+)-α-lipoic acid of the formula IV.

The examples below illustrate the invention.

### EXAMPLE 1

2.68 g (70 mmol) of sodium borohydride were introduced together with 1.00 g (2.4 mmol) of Aliquat 336^{(R)}, from Avocado Research Chemicals, in 150 ml of toluene and 21.8 g (100 mmol) of dimethyl (3S)-3-hydroxyoctanedioate. The reaction mixture was stirred at approximately 75° C. until complete conversion (monitored by TLC).

After the batch cooled to room temperature, methanol was added, the mixture was acidified with methanolic hydrochloric acid and the solvent mixture was distilled off. After addition of further methanol, the distillation was repeated.

After removing traces of solvent in vacuo, 16.2 g of methyl (6S)-6,8-dihydroxyoctanoate were obtained. This corresponds to a yield of 85%.

### Comparative Example 1

2.68 g (70 mmol) of sodium borohydride were introduced in 150 ml of toluene and 21.8 g (100 mmol) of dimethyl (3S)-3-hydroxyoctanedioate were added. The reaction mixture was stirred at approximately 75° C. until complete conversion (monitoring by TLC).

After the batch cooled to room temperature, methanol was added, the mixture was acidified with methanolic hydrochloric acid and the solvent mixture was distilled off. After addition of further methanol, the distillation was repeated.

After removal of traces of solvent in vacuo, 7.6 g of methyl (6S)-6,8-dihydroxyoctanoate were obtained. This corresponds to a yield of 40%.

1. A process for preparing dihydroxycarboxylic esters of the formula I, 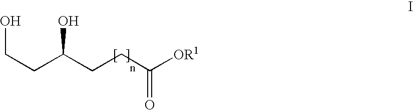
where
n is 1, 2, 3, 4, 5, 6 or 7 and
R¹ is an unsubstituted or substituted C₁-C₂₀-alkyl, C₂-C₂₀-alkenyl, C₂-C₂₀-alkynyl, C₃-C₈-cycloalkyl, aralkyl, aryl, hetarylalkyl or hetaryl radical,
which comprises
reacting hydroxycarboxylic diesters of the formula II, 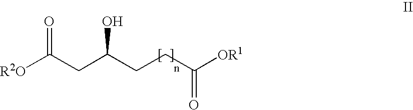
wherein R¹ is as defined above and
R² is a radical of an unsubstituted or substituted C₁-C₂₀-alkyl, C₂-C₂₀-alkenyl C₂-C₂₀-alkynyl, C₃-C₈-cycloalkyl, aralkyl, aryl, hetarylalkyl or hetaryl radical,
with at least one complex hydride in the presence of a solvent and in the presence of at least one phase transfer catalyst.

2. The process as claimed in claim 1, wherein the complex hydride is sodium borohydride.

3. The process as claimed in claim 1, wherein the solvent is an aprotic solvent.

4. The process as claimed in claim 1, wherein the solvent is toluene.

5. The process as claimed in claim 1, wherein the phase transfer catalyst is an ammonium salt of the formula III,
R³R⁴R⁵R⁶N⁺X⁻ III
where R³, R⁴, R⁵ and R⁶ are independently an unsubstituted or substituted aliphatic C₁-C₃₀ radical or an unsubstituted or substituted aralkyl or aryl radical and
X⁻ is a counterion.

6. A process for preparing R-(+)-α-lipoic acid of the formula IV 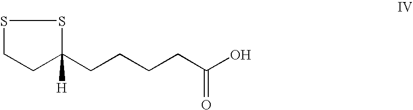
which comprises
preparing dihydroxycarboxylic esters of the formula I, 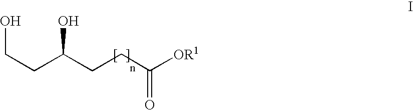
where
n is 3 and R¹ is an unsubstituted or substituted C₁-C₂₀-alkyl, C₂-C₂₀-alkenyl, C₂-C₂₀-alkynyl, C₃-C₈-cycloalkyl, aralkyl, aryl, hetarylalkyl or hetaryl radical
by reacting hydroxycarboxylic diesters of the formula II, 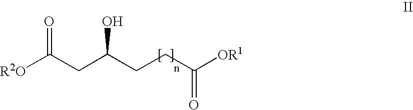
wherein R¹ is as defined above and
R² is a radical of an unsubstituted or substituted C₁-C₂₀-alkyl, C₂-C₂₀-alkenyl, C₂-C₂₀-alkynyl, C₃-C₈-cycloalkyl, aralkyl, aryl, hetarylalkyl or hetaryl radical,
with at least one complex hydride in the presence of a solvent and in the presence of at least one phase transfer catalyst and
a) converting the dihydroxycarboxylic esters of the formula I in organic solution using a sulfonyl chloride and a tertiary nitrogen base into the bissulfonic esters of I,
b) reacting the bissulfonic esters with sulfur and an alkali metal disulfide in a polar solvent to give the R-α-lipoic ester and
c) converting the R-α-lipoic ester into the R-(+)-α-lipoic acid of the formula IV.

7. The process as claimed in claim 2, wherein the solvent is an aprotic solvent.

8. The process as claimed in claim 7, wherein the phase transfer catalyst is an ammonium salt of the formula III,
R³R⁴R⁵R⁶N⁺X⁻ III
where R³, R⁴, R⁵ and R⁶ are independently an unsubstituted or substituted aliphatic C₁-C₃₀ radical or an unsubstituted or substituted aralkyl or aryl radical and X⁻ is a counterion.

9. The process as claimed in claim 4, wherein the phase transfer catalyst is an ammonium salt of the formula III,
R³R⁴R⁵R⁶N⁺X⁻ III
where R³, R⁴, R⁵ and R⁶ are independently an unsubstituted or substituted aliphatic C₁-C₃₀ radical or an unsubstituted or substituted aralkyl or aryl radical and X⁻ is a counterion.
